(19) United States
(12) Patent Application Publication
(10) Pub. No.: US 2018/0020944 A1
(43) Pub. Date: Jan. 25, 2018

(54) Stapedius Muscle Reflex Recording Electrode with a Sacrificial Part

(71) Applicant: MED-EL Elektromedizinische Geraete GmbH, Innsbruck (AT)

(72) Inventors: Andrzej Zarowski, Antwerpen (BE); Claude Jolly, Innsbruck (AT); Anandhan Dhanasingh, Chennai (IN)

(21) Appl. No.: 15/544,080

(22) PCT Filed: Jan. 19, 2016
(86) PCT No.: PCT/US16/13821

§ 371 Date: Jul. 17, 2017

Related U.S. Application Data
(60) Provisional application No. 62/105,260, filed on Jan. 20, 2015.

(51) Int. Cl. A61B 5/0492; A61N 1/36; A61B 5/00

(52) U.S. Cl. CPC A61B 5/0492; A61B 5/6848; A61N 1/36038

(57) ABSTRACT

A stapedius muscle recording electrode arrangement is described having one or more wire electrodes with an inner conducting wire covered by an outer layer of electrical insulation. There is an electrode opening in the electrical insulation that exposes underlying conducting wire. A curved needle has a tip configured for insertion into stapedius muscle tissue, and a base end coupled to the at least one wire electrode. The wire electrode and the needle are configured for insertion of the curved needle through the stapedius muscle tissue or between the stapedius muscle surface and the inner bony surface of the pyramidal eminence to embed the wire electrode in the stapedius muscle tissue or between the stapedius muscle surface and the inner bony surface of the pyramidal eminence for electrical interaction of the conducting wire at the electrode opening with the stapedius muscle tissue.

This application is a national phase entry of Patent Cooperation Treaty Application PCT/US2016/013821, which in turn claims priority from U.S. Provisional Patent Application 62/105,260, filed Jan. 20, 2015, both of which are incorporated herein by reference in their entireties.

### TECHNICAL FIELD

The present invention relates to an electrode configuration for insertion along or into the stapedius muscle.

### BACKGROUND ART

A normal ear transmits sounds as shown in FIG. 1 through the outer ear 101 to the tympanic membrane (eardrum) 102, which vibrates the ossicles of the middle ear 103 (malleus, incus, and stapes). The stapes footplate is positioned in the oval window 106 that forms an interface to the fluid filled inner ear (the cochlea) 104. Movement of the stapes generates a pressure wave in the cochlea 104 that stimulates the sensory cells of the auditory system (hair cells). The cochlea 104 is a long narrow duct wound spirally around its central axis (called the modiolus) for approximately two and a half turns. The cochlea 104 includes an upper channel known as the scala vestibuli, a middle channel known as the scala media and a lower channel known as the scala tympani. The hair cells connect to the spiral ganglion cells of the cochlear nerve 105 that reside in the modiolus. In response to received sounds transmitted by the middle ear 103, the fluid-filled cochlea 104 functions as a transducer to generate electric pulses which are transmitted to the cochlear nerve 105, and ultimately to the brain.

Hearing is impaired when there are problems in the ability to transduce external sounds into meaningful action potentials along the neural substrate of the cochlea 104. To improve impaired hearing, auditory prostheses have been developed. For example, when the impairment is related to operation of the middle ear 103, a conventional hearing aid or middle ear implant may be used to provide acoustic-mechanical stimulation to the auditory system in the form of amplified sound. Or when the impairment is associated with the cochlea 104, a cochlear implant with an implanted stimulation electrode can electrically stimulate auditory nerve tissue with small currents delivered by multiple electrode contacts distributed along the electrode.

FIG. 1 also shows some components of a typical cochlear implant system, including an external microphone that provides an audio signal input to an external signal processor 111 where various signal processing schemes can be implemented. The processed signal is then converted into a digital data format, such as a sequence of data frames, for transmission into the implant 108. Besides receiving the processed audio information, the implant 108 also performs additional signal processing such as error correction, pulse formation, etc., and produces a stimulation pattern (based on the extracted audio information) that is sent through an electrode lead 109 to an implanted electrode array 110.

Typically, the electrode array 110 includes multiple electrode contacts 112 on its surface that provide selective stimulation of the cochlea 104. Depending on context, the electrode contacts 112 are also referred to as electrode channels. In cochlear implants today, a relatively small number of electrode channels are each associated with relatively broad frequency bands, with each electrode contact 112 addressing a group of neurons with an electric stimulation pulse having a charge that is derived from the instantaneous amplitude of the signal envelope within that frequency band.

FIG. 2 shows various functional blocks in a signal processing arrangement for producing electrode stimulation signals to electrode contacts in an implanted cochlear implant array according to a typical hearing implant system. A pseudo code example of such an arrangement can be set forth as:

```
Input Signal Preprocessing:
  BandPassFilter (input_sound, band_pass_signals)
Envelope Extraction:
  BandPassEnvelope (band_pass_signals, band_pass_envelopes)
Stimulation Timing Generation:
  TimingGenerate (band_pass_signals, stim_timing)
Pulse Generation:
  PulseGenerate (band_pass_envelopes, stim_timing, out_pulses)
```

The details of such an arrangement are set forth in the following discussion.

In the arrangement shown in FIG. 2, the initial input sound signal is produced by one or more sensing microphones, which may be omnidirectional and/or directional. Preprocessor Filter Bank 201 pre-processes this input sound signal with a bank of multiple parallel band pass filters (e.g. Infinite Impulse Response (IIR) or Finite Impulse Response (FIR)), each of which is associated with a specific band of audio frequencies, for example, using a filter bank with 12 digital Butterworth band pass filters of 6th order, Infinite Impulse Response (IIR) type, so that the acoustic audio signal is filtered into some K band pass signals, U₁ to U_{K} where each signal corresponds to the band of frequencies for one of the band pass filters. Each output of sufficiently narrow CIS band pass filters for a voiced speech input signal may roughly be regarded as a sinusoid at the center frequency of the band pass filter which is modulated by the envelope signal. This is also due to the quality factor (Q≈3) of the filters. In case of a voiced speech segment, this envelope is approximately periodic, and the repetition rate is equal to the pitch frequency. Alternatively and without limitation, the Preprocessor Filter Bank 201 may be implemented based on use of a fast Fourier transform (FFT) or a short-time Fourier transform (STFT). Based on the tonotopic organization of the cochlea, each electrode contact in the scala tympani typically is associated with a specific band pass filter of the Preprocessor Filter Bank 201. The Preprocessor Filter Bank 201 also may perform other initial signal processing functions such as and without limitation automatic gain control (AGC) and/or noise reduction and/or wind noise reduction and/or beamforming and other well-known signal enhancement functions. An example of pseudocode for an infinite impulse response (IIR) filter bank based on a direct form II transposed structure is given by Fontaine et al., Brian Hears: Online Auditory Processing Using Vectorization Over Channels, Frontiers in Neuroinformatics, 2011; incorporated herein by reference in its entirety.

The band pass signals U₁ to U_{K} (which can also be thought of as electrode channels) are output to an Envelope Detector 202 and Fine Structure Detector 203. The Envelope Detector 202 extracts characteristic envelope signals outputs Y₁, . . . , Y_{K} that represent the channel-specific band pass envelopes. The envelope extraction can be represented by Yₖ=LP(|Uₖ|), where |.| denotes the absolute value and LP(.) is a low-pass filter; for example, using 12 rectifiers and 12 digital Butterworth low pass filters of 2nd order, IIR-type. Alternatively, the Envelope Detector 202 may extract the Hilbert envelope, if the band pass signals U₁, . . . , U_{K} are generated by orthogonal filters.

The Fine Structure Detector 203 functions to obtain smooth and robust estimates of the instantaneous frequencies in the signal channels, processing selected temporal fine structure features of the band pass signals U₁, . . . , U_{K} to generate stimulation timing signals X₁, . . . , X_{K}. The band pass signals U₁, . . . , Uₖ can be assumed to be real valued signals, so in the specific case of an analytic orthogonal filter bank, the Fine Structure Detector 203 considers only the real valued part of Uₖ. The Fine Structure Detector 203 is formed of K independent, equally-structured parallel sub-modules.

The extracted band-pass signal envelopes Y₁, . . . , Y_{K} from the Envelope Detector 202, and the stimulation timing signals X₁, . . . , X_{K} from the Fine Structure Detector 203 are input signals to a Pulse Generator 204 that produces the electrode stimulation signals Z for the electrode contacts in the implanted electrode array 205. The Pulse Generator 204 applies a patient-specific mapping function—for example, using instantaneous nonlinear compression of the envelope signal (map law)—That is adapted to the needs of the individual cochlear implant user during fitting of the implant in order to achieve natural loudness growth. The Pulse Generator 204 may apply logarithmic function with a form-factor C as a loudness mapping function, which typically is identical across all the band pass analysis channels. In different systems, different specific loudness mapping functions other than a logarithmic function may be used, with just one identical function is applied to all channels or one individual function for each channel to produce the electrode stimulation signals. The electrode stimulation signals typically are a set of symmetrical biphasic current pulses.

FIG. 3 shows a portion of the middle ear anatomy in greater detail, including the incus 301 and the stapes 302. The lenticular process end of the incus 301 vibrates the head 305 of the stapes 302, which in turn vibrates the base 303 of the stapes 302 which couples the vibration into the inner ear (cochlea). Also connected to the head 305 of the stapes 302 is the stapedial tendon 306 of the stapedius muscle situated within the bone of the pyramidal eminence 307. When a loud noise produces an excessively high sound pressure that could damage the inner ear, the stapedius muscle reflexively contracts to decrease the mechanical coupling of the incus 301 to the stapes 302 (and thereby also reduce the force transmission). This protects the inner ear from excessively high sound pressures.

The tensing of the stapedius muscle when triggered by such high sound pressures is also referred to as the stapedius reflex. Medically relevant information about the functional capability of the ear may be obtained by observation of the stapedius reflex. Measurement of the stapedius reflex also is useful for setting and/or calibrating cochlear implants because the threshold of the stapedius reflex is closely correlated to the psychophysical perception of comfortable loudness, the so-called maximal comfort level (MCL). The stapedius reflex can be determined in an ambulatory clinical setting using an additional device, an acoustic tympanometer that measures the changes in acoustic impedance of the middle ear caused by stapedial muscle contraction in response to loud sounds.

To measure the stapedius reflex intra-operatively, it is known to use electrodes that are brought into contact with the stapedius muscle to relay to a measuring device the action current and/or action potentials generated upon a contraction of the stapedius muscle. But a reliable minimally-invasive contact of the stapedius muscle is difficult because the stapedius muscle is situated inside the bony pyramidal eminence and only the stapedial tendon is accessible from the interior volume of the middle ear.

Various intraoperative stapedius muscle electrodes are known from U.S. Pat. No. 6,208,882, however, these only achieve inadequate contact of the stapedius muscle tissue (in particular upon muscle contraction) and are also very traumatizing. This reference describes one embodiment that uses a ball shape monopolar electrode contact with a simple wire attached to it. That would be very difficult to surgically position into a desired position with respect to the stapedius tissue and to fix it there allowing for a long-term atraumatic and stable positioning. Therefore the weakness of this type of electrode is that it does not qualify for chronic implantation. In addition, there is no teaching of how to implement such an arrangement with a bipolar electrode with electrode contacts with sufficient space between each other to enable bipolar registration.

Some intraoperative experiments and studies have been conducted with hook electrodes that have been attached at the stapedius tendon or muscle. These electrode designs were only suitable for acute intra-operative tests. Moreover, some single hook electrodes do not allow a quick and easy placement at the stapedius tendon and muscle—the electrode has to be hand held during intra-operative measurements, while other double hook electrodes do not ensure that both electrodes are inserted into the stapedius muscle due to the small dimensions of the muscle and the flexibility of the electrode tips. One weakness of these intraoperative electrodes is that they do not qualify for chronic implantation.

German patent DE 10 2007 026 645 (incorporated herein by reference) discloses a two-part bipolar electrode configuration where a first electrode is pushed onto the stapedius tendon or onto the stapedius muscle itself, and a second electrode is pierced through the first electrode into the stapedius muscle. One disadvantage of the described solution is its rather complicated handling in the very limited space of the surgical operation area, especially manipulation of the fixation electrode. In addition, the piercing depth of the second electrode is not controlled so that trauma can also occur with this approach. Also it is not easy to avoid galvanic contact between both electrodes.

U.S. Patent Publication 20100268054 (incorporated herein by reference) describes a different stapedius electrode arrangement having a long support electrode with a base end and a tip for insertion into the target tissue. A fixation electrode also has a base end and a tip at an angle to the electrode body. The tip of the fixation electrode passes perpendicularly through an electrode opening in the support electrode so that the tips of the support and fixation electrodes penetrate into the target tissue so that at least one of the electrodes senses electrical activity in the target stapedius tissue. The disadvantages of this design are analogous to the disadvantages mentioned in the preceding patent.

U.S. Patent Publication 20130281812 (incorporated herein by reference) describes a double tile stapedial electrode for bipolar recording. The electrode is configured to be placed over the stapedius tendon and a sharp tip pierces through the bony channel towards the stapedius muscle. The downside of this disclosure is again its rather complicated handling in the very limited space of the surgical operation area.

Various other stapedial electrode designs also are known, all with various associated drawbacks. A simple wire and ball contact electrode is very difficult to surgically position and to keep it atraumatically stabilized for chronic implantations. The penetrating tip of such a design must be stiff enough to pass through the bone tunnel, but if the tip is too stiff, it is difficult to bend and maneuver the wire into its position. And some stapedius muscle electrode designs are only monopolar electrodes (with a single electrode contact) and are not suitable for a bipolar arrangement with the electrode contacts with sufficient distance between each other to enable bipolar registration.

### SUMMARY

Embodiments of the present invention are directed to stapedius muscle recording electrode arrangements having one or more wire electrodes with an inner conducting wire covered by an outer layer of electrical insulation. There are one or more electrode openings in the electrical insulation that exposes underlying conducting wire. In some embodiments an extended portion of the conducting wire may be uninsulated to ensure the galvanic contact of the wire with the stapedial muscle tissue. A curved needle has a tip configured for insertion into or along the stapedius muscle tissue, and a base end coupled to the at least one wire electrode. The wire electrode and the curved needle are configured for insertion of the needle along or through the stapedius muscle tissue to position the conductive wire along the stapedial muscle or its tendon or to embed the wire electrode in the stapedius muscle tissue for electrical interaction of the conducting wire with the stapedius muscle tissue.

In some specific embodiments, the curvature of the curved needle may be constant over the entire needle or it may vary from a relatively larger curvature radius towards the tip of the needle to a relatively smaller curvature radius towards the base end of the needle. There may be a transition section of non-metallic material or an isolated metallic wire that couples the base end of the curved needle to the at least one wire electrode. This transition section may be malleable. There may be a ball shaped electrode contact at each electrode opening connected to the underlying conducting wire and extending out through the electrode opening above the outer layer of electrical insulation. There may be a drug eluting component incorporated into the electrical insulation and configured to release a therapeutic drug over time from the embedded at least one wire electrode into adjacent stapedius muscle tissue. In some embodiments there may be two wire electrodes configured for bipolar operation. The at least one wire electrode and the curved needle may possess a single shared longitudinal axis. And the curved needle may have a stiffness greater than that of the at least one wire electrode.

Embodiments of the present invention also include methods for embedding a stapedius muscle electrode along or into the stapedius muscle tissue of a patient. A stapedius muscle electrode according to any of the above arrangements is provided. An opening is drilled into the bone of the pyramidal eminence of the patient at least part way towards the underlying stapedius muscle. Then—if positioning of the electrode along (not within) the stapedial muscle is chosen—a tunneling instrument may be used to perform a tunnel between the opening drilled in the pyramidal eminence and the natural orifice of the stapedial tendon. The tunnel is created between the muscle and the inner bony surface of the pyramidal eminence. The tip of the curved needle is then inserted through the opening in the pyramidal eminence into the stapedius muscle. The curved needle is then directed into and through the tunnel and out of the natural orifice of the stapedial tendon or it is directed through the stapedius muscle into the stapedius tendon and out at the distal end (i.e. the end towards the stapes) of the stapedius tendon. The curved needle is pulled out along the outer surface of the stapedius tendon close to the head of the stapes to embed the at least one wire electrode and the electrode opening in the stapedius muscle or along the tunnel. Then the curved needle is separated from the at least one wire electrode or from the transition section. Opposite direction of electrode positioning is also possible.

The opening may have a diameter of 0.5 mm and the tunnel may have a diameter of 100-200 μm. The curved needle may be separated from the at least one wire electrode or from the transition section at the distal end of the stapedius tendon, or at a distance away from the distal end of the stapedius tendon so as to leave a section of the wire electrode to be secured against the pyramidal eminence to fix the at least one wire electrode into position embedded in the stapedius muscle or in the tunnel. This fixation may be achieved by bending the wire over the bony rim of the pyramidal eminence.

### BRIEF DESCRIPTION OF THE DRAWINGS

FIG. 1 shows anatomical structures of a typical human ear.

FIG. 2 shows various functional blocks in a signal processing arrangement for a typical cochlear implant system

FIG. 3 shows detailed anatomy around the stapedius tendon in a human ear.

FIG. 4 A-C shows stapedius electrode arrangements according to various specific embodiments of the present invention.

FIG. 5 A-F shows various steps in implanting a stapedius electrode according to an embodiment of the present invention.

FIG. 6 shows an alternative electrode arrangement.

FIG. 7 shows an alternative electrode arrangement with a second recording electrode and a separate movable fixation element.

FIG. 8 shows an alternative electrode arrangement with a second recording electrode mounted on a movable element.

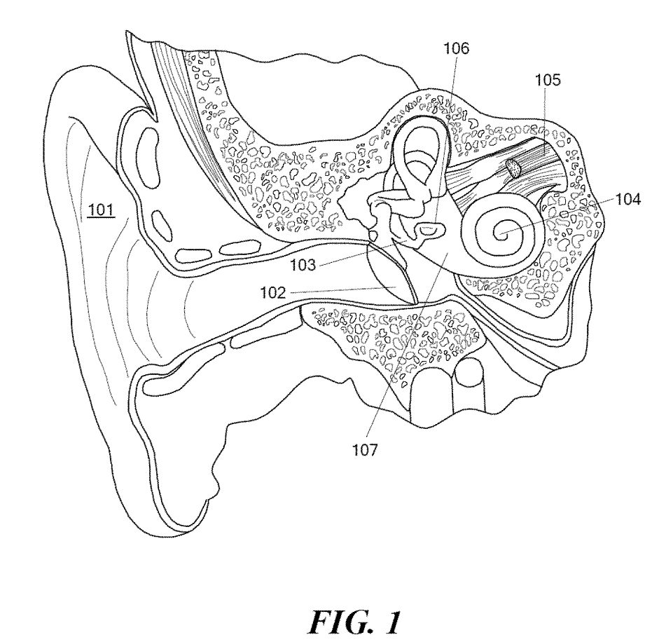

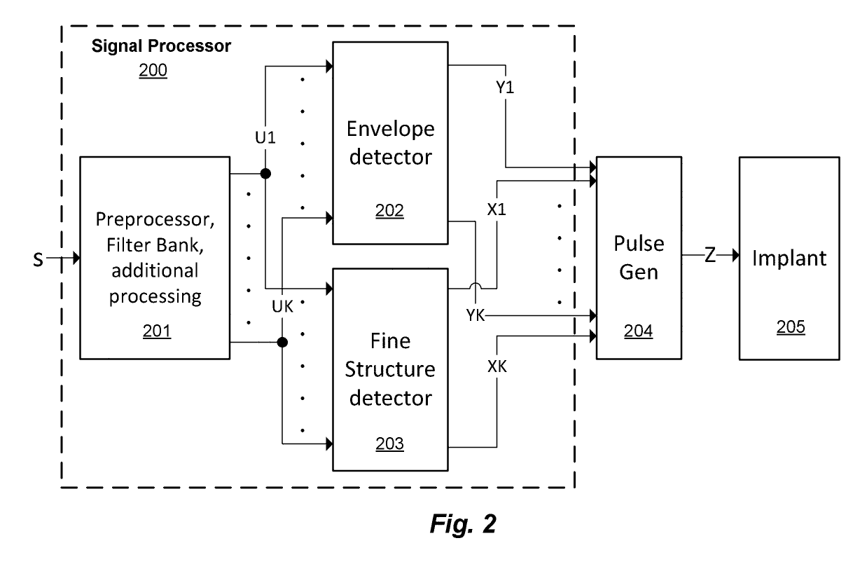

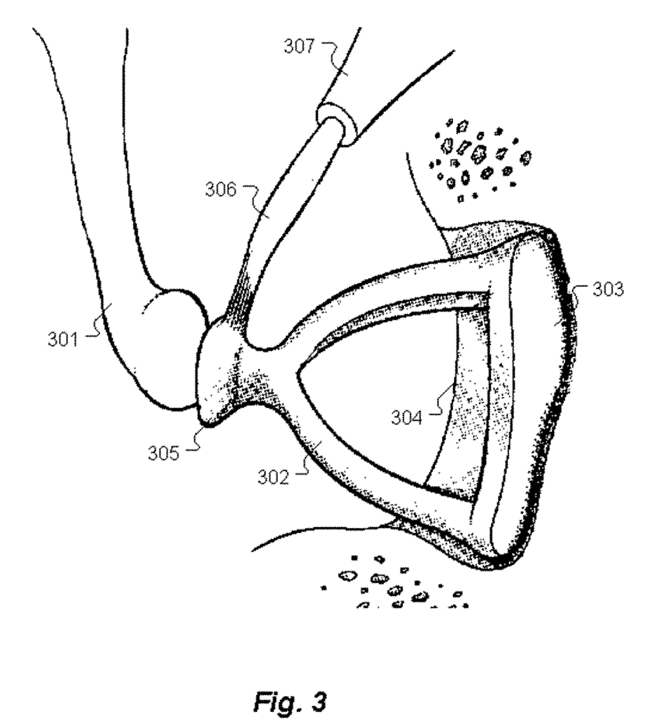

### DETAILED DESCRIPTION

Various embodiments of the present invention are directed to stapedius muscle recording electrode arrangements that use a simple inexpensive electrode (e.g. wire electrode) that is attached to a curved needle to be passed inside the pyramidal eminence between a surgically created opening in the pyramidal eminence and the natural orifice of the stapedial tendon.

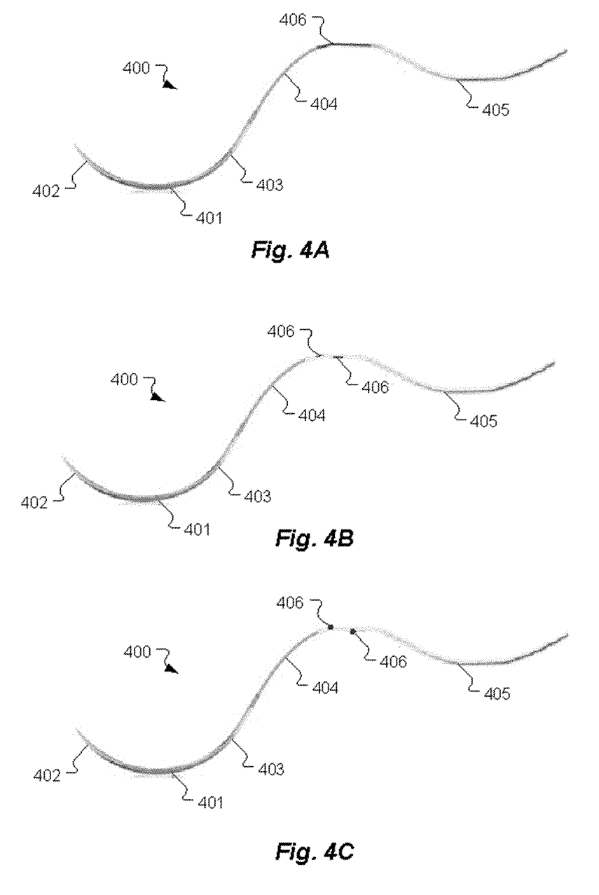

FIG. 4 A-C shows stapedius electrode arrangements 400 according to various specific embodiments of the present invention. As seen in the figures, there are one or more wire electrodes 405 with an inner conducting wire covered by an outer layer of electrical insulation such as silicone. For example, the inner conducting wire may be 50μ diameter platinum wire. In some specific embodiments, there may be a drug eluting component incorporated into the electrical insulation of the at least one wire electrode 405 and configured to release a therapeutic drug over time from the embedded at least one wire electrode 405 into adjacent stapedius muscle tissue. In each wire electrode 405 there is an electrode opening 406 in the electrical insulation that exposes underlying conducting wire to form an electrode contact for electrical interaction of the wire electrode 405 with the stapedius muscle tissue. The far end of the electrode wire 405 may be attached to any device for processing the recorded electrical potentials from the stapedius muscle.

In the embodiments shown in FIGS. 4 A-C, the base end 403 of the curved needle 401 is coupled to the at least one wire electrode 405 by a transition section of suture material 404 that may be conductive or non-conductive; for example, 25μ diameter platinum wire 1-5 mm in length (e.g. 2.5 mm) may be used. Suture material 404 also may be malleable. In other specific embodiments, suture material may be omitted and the curved needle 401 may be directly coupled to at least one wire electrode 405. FIG. 4A shows an embodiment with just one wire electrode 405 for monopolar operation. There is an electrode opening 406 for monopolar recording. The electrode opening 406 may have a length of 1-10 mm (e.g. 8 mm) and along this length the entire wire surface may be uninsulated. FIGS. 4 B-C show embodiments with two wire electrodes 405 configured for bipolar operation. In the embodiment shown in FIG. 4B, there are two electrode openings 406 that are offset from each other by an appropriate distance for bipolar recording. FIG. 4C shows an embodiment with ball-shaped electrode contacts at each electrode opening 406, which are connected to the underlying conducting wire and extending out through the electrode opening 406 above the outer layer of electrical insulation that forms the outer surface of the at least one wire electrode 405.

A curved needle 401 has a tip 402 configured for insertion into stapedius muscle tissue, and a base end 403 coupled to the at least one wire electrode 405 or to the transition section 404. Typically, the curvature of the curved needle 401 may be constant over the entire needle or it may vary from a relatively larger curvature radius towards the tip of the needle to a relatively smaller curvature radius towards the base end of the needle. Further a typical length of the curved needle may be 2-3 mm and a typical thickness may be 50-100 μm. The at least one wire electrode 405 and the curved needle 401 are configured for insertion of the needle 401 through the stapedius muscle tissue to embed the wire electrode 405 in the stapedius muscle tissue or through the tunnel. The at least one wire electrode 405 and the curved needle 401 may possess a single shared longitudinal axis. And the curved needle 401 may have a stiffness greater than that of the at least one wire electrode 405.

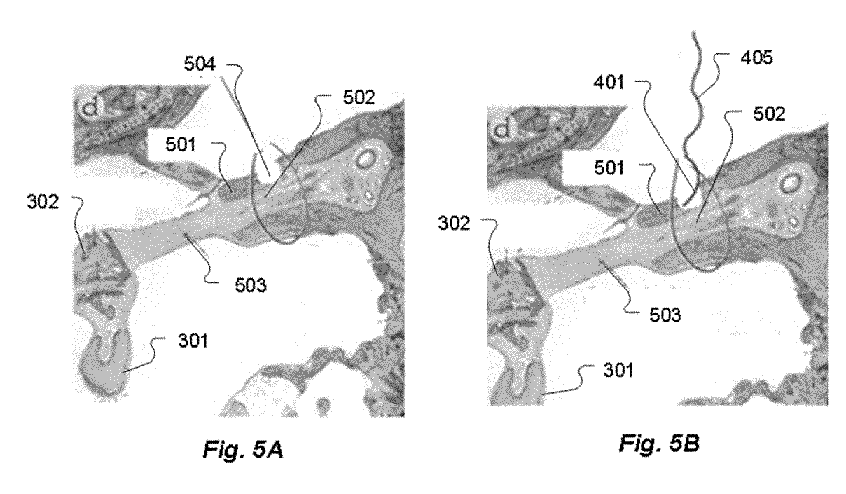

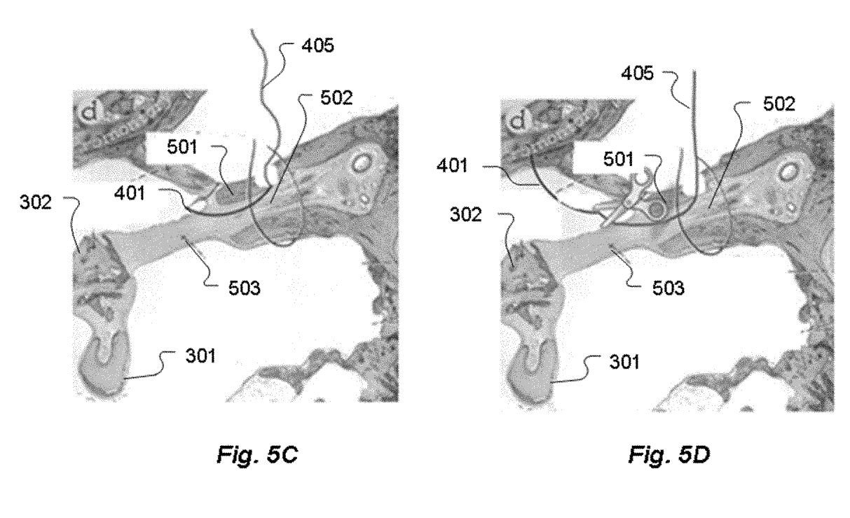

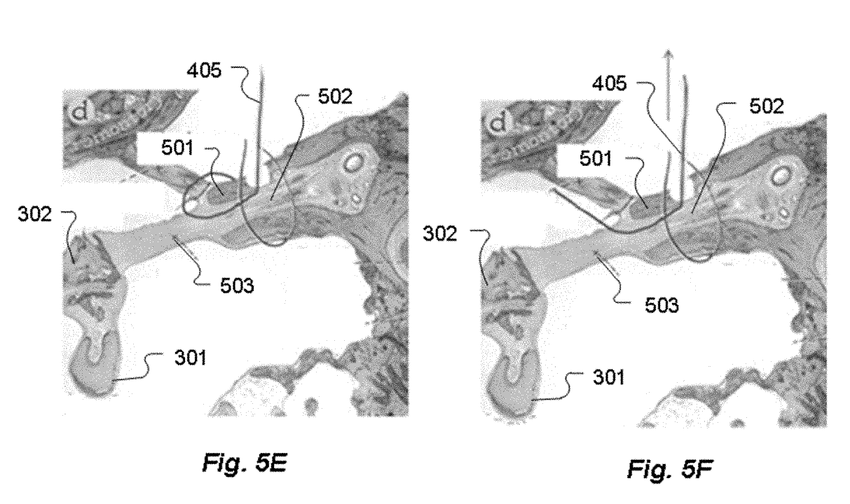

FIGS. 5 A-F show various steps in implanting a stapedius electrode arrangement 400. Initially as shown in FIG. 5A, the surgeon drills an opening 504 into the bone of the pyramidal eminence 501 of the patient's temporal bone, at least part way towards the underlying stapedius muscle 502. Then—if positioning the electrode 400 along (not within) the stapedial muscle 502 is chosen—a tunneling instrument may be used to create a tunnel between the opening drilled in the pyramidal eminence 501 and the natural orifice of the stapedial tendon 503. In this case the stapedius muscle 502 should first be dissected from the inner bony surface of the pyramidal eminence 501. This can be achieved using the tunneling tool. Typically, the opening 504 may have a diameter of about 0.5 mm, and the diameter of the curved needle 401 and the at least one wire electrode 405 would be at least slightly smaller to fit into the opening 504. The tip 402 of the curved needle 401 is inserted through the opening 504 in the pyramidal eminence 504 into the stapedius muscle 502, as shown in FIG. 5B, or into the tunnel and then directed through the stapedius muscle 502 and into the stapedius tendon 503 or passing through the tunnel. In either case, the curved needle 401 exits at or near the distal end of the stapedius tendon 503, as shown in FIG. 5C. The curved needle 401 is pulled out along the outer surface of the stapedius tendon 503 close to the head of the stapes to embed the at least one wire electrode 405 and the electrode opening 406 in the stapedius muscle 502 or along the tunnel.

Then once the wire electrode 405 has assumed its final position, as shown in FIG. 5D, the curved needle 401 is separated from the at least one wire electrode 405, for example, by cutting. The curved needle 401 may be separated from the at least one wire electrode 405 right at the distal end of the stapedius tendon 503, or at a distance away from the distal end of the stapedius tendon 503, as shown in FIG. 5E, so as to leave a section of the wire electrode 405 to be bent against the bony rim of the pyramidal eminence 501 to fix the position of the at least one wire electrode 405 that is embedded in the stapedius muscle or in the tunnel. In such an embodiment, the wire electrode 405 may still be easily explanted when necessary, as shown in FIG. 5F, simply by unbending the length that is coiled about the pyramidal eminence 501.

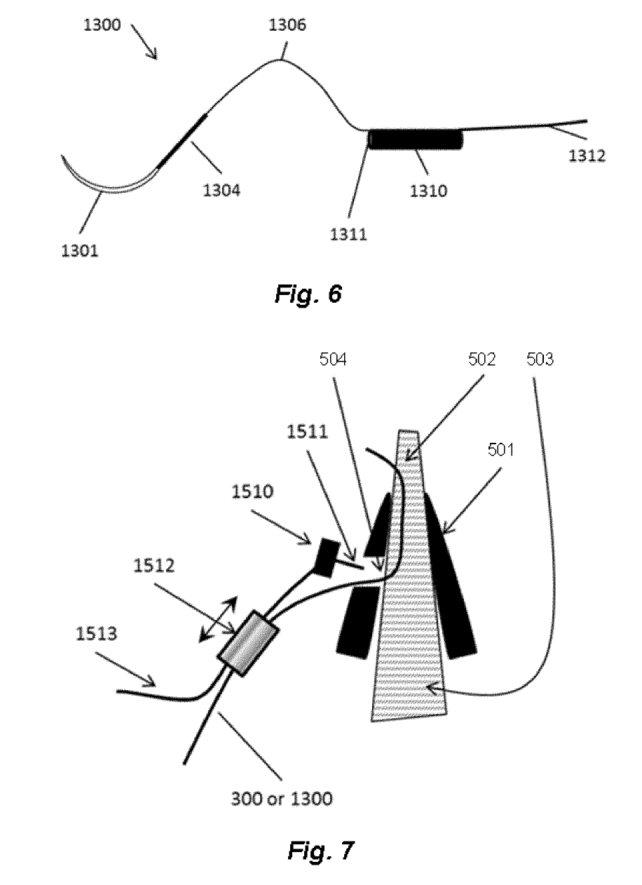

In alternative embodiments an electrode arrangement 1300 as shown in FIG. 6 may be used. This electrode arrangement 1300 includes a curved needle 1301, a transition section 1304 and a wire electrode 1306. These components are comparable to the corresponding components described with reference to electrode arrangement 400 above. Here, the entire wire electrode 1306 is shown without insulation as a different example to the above. At the proximal end of wire electrode 1306, a cylindrical section 1310 is attached. The cylindrical section 1310 may be made of any conductive biocompatible material and may typically have a length of 1-2 mm, corresponding to a typical length of an opening 504 into the bone of the pyramidal eminence 501 of the patient's temporal bone. Similarly, the thickness is typically about 0.5 to 1 mm, again corresponding to the thickness of the drilled opening 504. The entire surface of the cylindrical section 1310 may be electrically insulated except the surface 1311 which may remain electrically conductive and which attaches to the wire electrode 1306. During insertion of the electrode arrangement 1300, surface 1311 may be advance into opening 504 far enough to attach to stapedius tissue. That way, the surface 1311 may increase the electrically conductive area and contribute to an increased sensitivity of the recording arrangement. The opposite end of cylindrical section 1310 may be attached to lead 1312, which in turn may be attached to any device for processing the recorded electrical potentials from the stapedius muscle. As an alternative to the cylindrical form, section 1310 may have another geometrical form, e.g. it may have a ball shaped section.

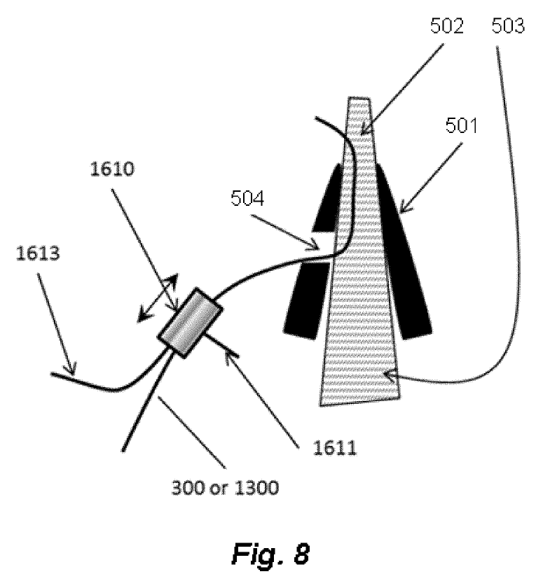

Alternative embodiments of electrode arrangements (preferably bipolar recording arrangements), are shown in FIGS. 7 and 8 where electrode arrangements 400 or 1300 may be used again, with other additional components. In FIG. 7, a separate electrically isolated conductor 1513 may have at its distal end a terminal port 1510 from which a recording electrode 1511 protrudes. Recording electrode 1511 may be inserted into stapedius tissue 502/503 through opening 504. A movable hollow element 1512 formed, for example, as a cylinder, may hold leads 1312 and 1513 in close proximity to each other and provide a fixation means for the entire electrode arrangement. Alternatively, as shown in FIG. 8, recording electrode 1611 may protrude directly from movable element 1610. The size of the moveable element 1610 and the terminal port 1510 may be comparable to the opening 504 such that they can snuggly fit into the opening 504. Alternatively, they may be larger in size such that the recording electrodes 1611 and 1511 may have a defined insertion depth into the stapedial tissue.

The electrode arrangements 400 or 1300 or the alternative arrangements shown in FIGS. 7 and 8 may be part of a cochlear implant system or any other implantable system which can take advantage of a signal recorded from the stapedius muscle tissue. Branches 1312, 1513, 1613 or the far end of electrode wire 405 may be attached either directly to electronic circuitry within an implantable stimulator or it may be attached to an electrode branch as described e.g. in US3005216073, incorporated herein by reference.

For the monopolar recording configuration via electrode arrangements 400 or 1300 any reference electrode provided by the implantable device used may be exploited. Alternatively, a separate reference electrode, e.g. placed subperiosteally in the proximity of the ear may be used.

Although various exemplary embodiments of the invention have been disclosed, it should be apparent to those skilled in the art that various changes and modifications can be made which will achieve some of the advantages of the invention without departing from the true scope of the invention.

What is claimed is:

1. A stapedius muscle electrode arrangement comprising:
at least one wire electrode having an inner conducting wire covered by an outer layer of electrical insulation with an electrode opening that exposes underlying conducting wire; and
a curved needle having:
i. a tip configured for insertion into contact with stapedius muscle tissue, and
ii. a base end coupled to the at least one wire electrode;
wherein the at least one wire electrode and the curved needle are configured for insertion of the needle through or along the stapedius muscle tissue to embed the at least one wire electrode into galvanic contact with the stapedius muscle tissue to provide for electrical interaction of the conductive wire with the stapedius muscle tissue.

2. The electrode arrangement according to claim 1, wherein a section of suture material couples the base end of the curved needle to the at least one wire electrode.

3. The electrode arrangement according to claim 1, further comprising:
a ball shaped electrode contact at each electrode opening connected to the underlying conducting wire and extending out through the electrode opening above the outer layer of electrical insulation.

4. The electrode arrangement according to claim 1, further comprising:
a drug eluting component incorporated into the electrical insulation and configured to release a therapeutic drug over time from the embedded at least one wire electrode into adjacent stapedius muscle tissue.

5. The electrode arrangement according to claim 1, wherein there are two wire electrodes configured for bipolar operation.

6. The electrode arrangement according to claim 1, wherein the at least one wire electrode and the curved needle possess a single shared longitudinal axis.

7. The electrode arrangement according to claim 1, wherein the curved needle has a stiffness greater than that of the at least one wire electrode.

8. A method of embedding a stapedius muscle electrode into stapedius muscle tissue, the method comprising:
providing a stapedius muscle electrode arrangement including:
i. at least one wire electrode having an inner conducting wire covered by an outer layer of electrical insulation with an electrode opening that exposes underlying conducting wire; and
ii. a curved needle having a tip configured for insertion into stapedius muscle tissue, and a base end coupled to the at least one wire electrode;
drilling an opening into bone of the pyramidal eminence of the patient at least part way towards the underlying stapedius muscle;
creating a tunnel between the opening and a natural orifice of the stapedial tendon;
inserting the tip of the curved needle through the tunnel into the stapedius muscle;
directing the tip of the curved needle through the stapedius muscle into the stapedius tendon and out the distal end of the stapedius tendon;
pulling the curved needle out through the outer surface of the stapedius tendon to embed the at least one wire electrode and the electrode opening in the stapedius muscle; and
separating the curved needle from the at least one wire electrode.

9. The method according to claim 8, wherein a section of suture material couples the base end of the curved needle to the at least one wire electrode.

10. The method according to claim 8, further comprising:
a ball shaped electrode contact at each electrode opening connected to the underlying conducting wire and extending out through the electrode opening above the outer layer of electrical insulation.

11. The method according to claim 8, wherein a drug eluting component is incorporated into the electrical insulation and configured to release a therapeutic drug over time from the embedded at least one wire electrode into adjacent stapedius muscle tissue.

12. The method according to claim 8, wherein there are two wire electrodes configured for bipolar operation.

13. The method according to claim 8, wherein the tunnel has a diameter of 0.5 mm.

14. The method according to claim 8, wherein the curved needle is separated from the at least one wire electrode at the distal end of the stapedius tendon.

15. The method according to claim 8, wherein the curved needle is separated from the at least one wire electrode at a distance away from the distal end of the stapedius tendon so as to leave a section of the wire electrode, and wherein the method further comprises securing the section of the wire electrode against the pyramidal eminence to fix the at least one wire electrode into position embedded in the stapedius muscle or along the outer surface of the stapedius muscle.

16. The method according to claim 15, where the fixation is achieved by bending of the wire over the bony rim of the pyramidal eminence between the tunnel and the natural orifice of the stapedial tendon.

17. The method according to claim 8, wherein the at least one wire electrode and the curved needle possess a single shared longitudinal axis.

18. The method according to claim 8, wherein the curved needle has a stiffness greater than that of the at least one wire electrode.
